(19) United States
(12) Patent Application Publication
(10) Pub. No.: US 2022/0348533 A1
(43) Pub. Date: Nov. 3, 2022

(54) METHOD FOR THE PURIFICATION OF ANILINE

(71) Applicants: COVESTRO INTELLECTUAL PROPERTY GMBH & CO. KG, LEVERKUSEN (DE); COVESTRO DEUTSCHLAND AG, LEVERKUSEN (DE)

(72) Inventors: Friedhelm Steffens, Leverkusen (DE); Jonas Krause, Langenfeld (DE); Denise Chan, Düsseldorf (DE)

(21) Appl. No.: 17/620,900

(22) PCT Filed: Jul. 1, 2020
(86) PCT No.: PCT/EP2020/068525

§ 371 Date: Dec. 20, 2021

(30) Foreign Application Priority Data
Jul. 3, 2019 (EP) 19184067.7
Jun. 15, 2020 (EP) 20180092.7

(51) Int. Cl. C07C 209/84

(52) U.S. Cl. CPC C07C 209/84

(57) ABSTRACT

The invention relates to a method for the purification of aniline, comprising the following steps: a) providing a raw aniline fraction; b) extracting the raw aniline fraction with an aqueous extractant containing, in relation to the total mass of the aqueous extractant, an alkali metal hydroxide in a concentration range from 0.009 to 2.05 mass % and an alkali metal salt that is different from an alkali metal hydroxide in a concentration range from 2.40 to 25.0 mass %, wherein an organic aniline phase and an aqueous amino phenolate phase are obtained after a phase separation; c) distilling the organic aniline phase from step b), obtaining a flow of purified aniline, a gaseous flow that boils at a lower temperature than aniline and containing organic impurities, and a liquid flow that boils at a higher temperature than aniline and containing organic impurities and aniline.

The present invention relates to a process for the purification of aniline, comprising the steps of: a) providing a crude aniline fraction; b) extracting the crude aniline fraction with an aqueous extractant containing, based on the total mass of the aqueous extractant, an alkali metal hydroxide in a concentration range from 0.009% by mass to 2.05% by mass and an alkali metal salt different from an alkali metal hydroxide in a concentration range from 2.40% by mass to 25.0% by mass, wherein, after phase separation, an organic aniline phase and an aqueous aminophenolate phase are obtained, and c) distilling the organic aniline phase from step b) to obtain a stream of purified aniline, a gaseous stream containing organic impurities having a lower boiling point than aniline, and a liquid stream containing aniline and organic impurities having a higher boiling point than aniline.

Aniline is an important intermediate for example for the preparation of di- and polyisocyanates of the diphenylmethane series (MDI) and is generally produced on the industrial scale by the catalytic hydrogenation of nitrobenzene in the gas or liquid phase (see, e.g., EP-A-0 696 573, EP-A-0 696 574, EP-A-1 882 681 and WO 2013/139737 A1). In this reaction, in addition to the target product aniline, secondary components such as for example phenol or aminophenols are also formed and must be removed prior to further use of the aniline in downstream processes. A particular area of concern is the removal of such secondary components whose boiling points are very similar to those of the target product to be produced because in this case distillation is considerably laborious. In the case of the production of aniline (boiling point at standard pressure: 184° C.), the removal of phenol (boiling point at standard pressure: 182° C.) in particular represents a major challenge for distillation technology, which is reflected in the use of long distillation columns with a large number of separating stages and high reflux ratios and accordingly high energy demand. A vacuum distillation for the removal of phenol from aniline is described for example in DE-A-1935363.

For this reason, there has been no shortage of approaches for separating phenol (and other phenolic compounds) from aniline in other ways, in particular by conversion to phenolate salts by reaction with suitable bases. As non-volatile, readily water-soluble compounds, the phenolate salts formed by this are substantially easier to separate from aniline than the phenolic compounds themselves.

For instance, JP-A-49-035341 describes a process in which aniline is brought into contact with solid alkali metal hydroxides in a fixed bed and only then sent to the distillation, or in which the distillation is conducted in the presence of the solid alkali metal hydroxide in proportions of from 0.1% to 3% by mass, based on the amount of aniline to be distilled. This simplifies the removal of critical components such as the aminophenols. However, this process has the disadvantages that large molar excesses of the solid alkali metal hydroxides are used with respect to the acidic secondary components to be removed and that it is not possible to precisely dose the alkaline compounds. On the one hand, in the case of overdosing, this can lead to corrosion problems, failures and highly viscous bottom phases in the distillation column, and on the other hand, in the case of underdosing, can lead to incomplete removal of the critical components.

EP-B-1 670 747 describes a process for separating compounds having phenolic hydroxyl groups from aromatic amines, wherein prior to the distillation a base is added to the amine to be purified in a molar ratio of 0.5:1 to 10:1, based on the phenolic compounds, optionally in the presence of polyols, such as for example polyethylene glycol. However, especially for aniline, which is produced in very great amounts, such an addition of polyols is economically disadvantageous and there is the risk of contaminating the product with polyol (fragments). Without the use of such a polyol, however, frequent process interruptions because of the precipitation of salts should be expected.

EP-A-1 845 079 describes a process for the purification of aniline by addition of an aqueous alkali metal hydroxide solution before or during the distillation, where the problems caused by deposition of solids, fouling and/or intense viscosity increase during the distillation are prevented by partially discharging the bottom phase of the distillation, washing it with water or dilute alkali metal hydroxide solution, and recycling the washed organic phase back into the distillation. The disadvantage here is the need for an additional process step to maintain reliable operation. In addition, this process produces an additional, organically polluted waste water stream that has to be worked up and disposed of.

EP-A-2 028 176 describes a process for purifying aromatic amines, in which the crude amine obtained after removal of the process water is treated with aqueous alkali metal hydroxide solution and the process product thus obtained is distilled. The bottom product of the distillation column is partially to completely discharged and to some extent evaporated via two series- or parallel-connected evaporators (E¹) and (E²). The aim of this is to achieve maximum depletion of the valuable amine in the bottoms of the distillation column with minimal apparatus expense and energy consumption. In the aforementioned processes, the aromatic amine is distilled in the presence of a base. In this procedure, problems caused by corrosion, deposition of solids and/or fouling during the distillation have to be prevented by complex and/or expensive measures. Moreover, such processes are not well suited to the purification of crude aniline having high aminophenol contents since the aminophenolates formed during the reaction with base are only soluble in aniline in very small proportions and hence form solids which lead to fouling and deposits in the following aniline column.

Also known are those processes which remove the phenol and other phenolic compounds by means of an extraction with aqueous base. In order to also remove other, non-acidic impurities, the aniline nevertheless has to be distilled at least to some extent. Such processes are described for example in EP-A-1 845 080, EP-A-2 263 997, JP-A-08-295654 or else in EP-A-2 641 892. Among these, EP-A-1 845 080 will be considered in more detail by way of example. This document describes a process in which the choice of the concentration of the alkali metal hydroxide solution used for the extraction and of the temperature ensures that the aqueous phase is always the lower phase in the subsequent phase separation. In this way, phase separation problems, in particular phase inversion, are avoided during the extraction. Preferred alkali metal hydroxide concentrations are between 0.71 and 35 percent by weight. However, higher concentrations, such as for example 50 percent by weight, are also described. Lower concentrations, for example up to 0.1 percent by weight, can also be used when it is ensured by a sufficiently high temperature (up to 140° C., preferably up to 100° C., particularly preferably up to 95° C.) that the aqueous phase is always the lower phase. In each of the examples the extraction was conducted at 90° C. There must therefore be either a sufficiently high alkali metal hydroxide concentration or a sufficiently high temperature, both of these being associated with costs. The patent application does not reveal how the density differences between the aqueous phase and the organic phase that are required for good phase separation can be achieved without these measures.

According to the teaching of EP-A-1 845 080, the alkali metal hydroxide solution used for the extraction can be recycled after the extraction, possibly after additional purification and/or concentration, and reused for the extraction. It cannot be gathered from the document how this can specifically be done, in particular how the alkali metal hydroxide solution which is contaminated with organic impurities (especially phenolate) can be economically purified so that problem-free recycling is possible. As an alternative to recycling into the extraction, the document discloses supplying the alkali metal hydroxide solution used for the extraction, possibly after additional purification, to a waste water stream which is sent for example after subsequent workup to a water treatment plant. Here, losses of alkali metal hydroxide are inevitable. In summary, it can therefore be stated that, while this patent application does disclose a process which makes it possible to reliably ensure that the aqueous phase is always the lower phase (prevention of phase inversion), the price of this is the disadvantage of a high alkali metal hydroxide concentration or a high extraction temperature. Furthermore, there is no practicable concept for reusing excess alkali metal hydroxide.

JP 3804082 B2 describes a process for the purification of phenol-containing aniline, in which aniline is extracted with dilute alkali metal hydroxide solution. Preferably, a concentration of alkali metal hydroxide in the aqueous phase, after mixing of aniline and alkali metal hydroxide solution, is set in the range from 0.1% to 0.7% by mass. The molar ratio of alkali metal hydroxide to phenols is preferably in the range from 3 to 500.

In order to meet contemporary purity requirements, two or more extraction stages are generally required, as described for example in JP-A-2007217405, which further increases complexity. In addition, all of these processes produce large phenolate-containing waste water streams which in turn have to be carefully worked up and disposed of.

JP-A-2005 350388 very generally deals with improving the aniline workup. A process is described in which part of the bottoms of the aniline distillation column is discharged from said column and is converted to the gas phase separately, that is to say in a second evaporator different from the actual evaporator of the column. The gas phase thus obtained is recycled into the pure aniline column; heavy boiler fractions that cannot be evaporated are removed. The disadvantage of this process is that low boilers and water have to be removed separately in a process complex in terms of equipment in a dewatering column by an additional distillation that is upstream of the actual aniline distillation column.

Lastly, EP-A-1 602 640 describes a process in which the aqueous amine solution is purified in two distillation columns connected in series. One of the columns is operated at 2 to 20 bar and the other at 0.1 to 10 bar. The heat of condensation of the vapors emerging from the column operated at higher pressure is used here to heat the bottom of the column operated at lower pressure. The amine is obtained as bottom product. Accordingly, the process is not suitable for removing high boilers (such as the aminophenols) or phenol from aniline. Because of the high pressure, high bottom temperatures are also necessary, which can lead to some degree of decomposition of the product and hence to losses of yield or fouling.

There was therefore a need for further improvements in the area of aniline purification. Where it concerns removal of the phenolic compounds, attention has to date primarily been focused on phenol itself and less so on aminophenols. The known processes using a base have the disadvantages outlined above when purifying crude aniline fractions with high aminophenol contents. When using a base in the distillation, the recited problem of the formation of solids arises. If a base is dispensed with, the removal of the phenol which is regularly also present is difficult. When using an alkaline extraction, large amounts of base are required. The concentration and/or the amount of the base solution to be used—usually sodium or potassium hydroxide—cannot be arbitrarily reduced without impairing the extraction efficiency and/or the following phase separation.

It has been found, completely surprisingly, that the mentioned problems can be solved or at least greatly mitigated if a base extraction is placed upstream of the distillation of the crude aniline, in which base extraction, in addition to an alkali metal hydroxide used as basic component, an alkali metal salt different from an alkali metal hydroxide is also used and in particular the aqueous phase obtained after phase separation is subjected after acidification to a back-extraction with an organic stream such as a high-boiler-rich bottom stream obtained in the distillation.

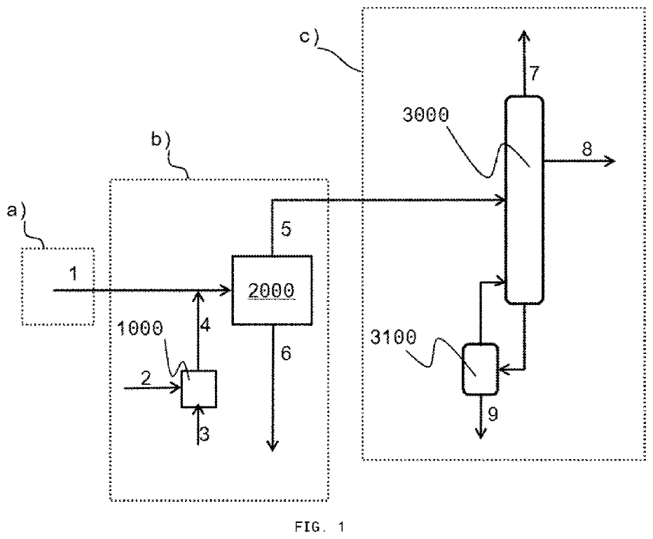

Taking account of the outlined need, a subject of the present invention is therefore a process for the purification of aniline, comprising the following steps (cf. also FIG. 1):
a) providing a crude aniline fraction (1) (comprising aqueous and organic constituents) containing aniline and organic impurities, wherein the organic impurities comprise phenol, organic impurities having a lower boiling point than aniline (referred to as “low boilers”), and aminophenol, and also further organic impurities having a higher boiling point than aniline (referred to as “high boilers”), and the concentration of aminophenol, based on the total mass of the organic constituents of the crude aniline fraction, is in the range from 0.001% by mass to 1.00% by mass, preferably in the range from 0.001% by mass to 0.50% by mass, particularly preferably in the range from 0.001% by mass to 0.10% by mass;
b) extracting the crude aniline fraction from step a) with an aqueous extractant (4) containing, based on the total mass of the aqueous extractant,
an alkali metal hydroxide, especially sodium or potassium hydroxide, in a concentration range from 0.009% by mass to 2.05% by mass, preferably 0.010% by mass to 2.00% by mass, particularly preferably 0.010% by mass to 1.00% by mass, very particularly preferably 0.50% by mass to 1.00% by mass, extremely particularly preferably 0.76% by mass to 1.00% by mass,
and
an alkali metal salt different from an alkali metal hydroxide, especially sodium or potassium chloride or sodium or potassium sulfate, in a concentration range from 2.40% by mass to 25.0% by mass, preferably 4.00% by mass to 25.0% by mass, particularly preferably 5.00% by mass to 25.0% by mass, very particularly preferably 10.0% by mass to 20.0% by mass, extremely particularly preferably 12.0% by mass to 17.0% by mass,
wherein, after phase separation, an organic aniline phase (5) depleted in aminophenol (and as a matter of course in phenol as well) and an aqueous aminophenolate phase (6) are obtained; and
c) distilling the organic aniline phase (6) from step b) to obtain a stream of purified aniline (8), a gaseous stream (7) containing organic impurities having a lower boiling point than aniline, and a liquid stream (9) containing aniline and organic impurities having a higher boiling point than aniline;

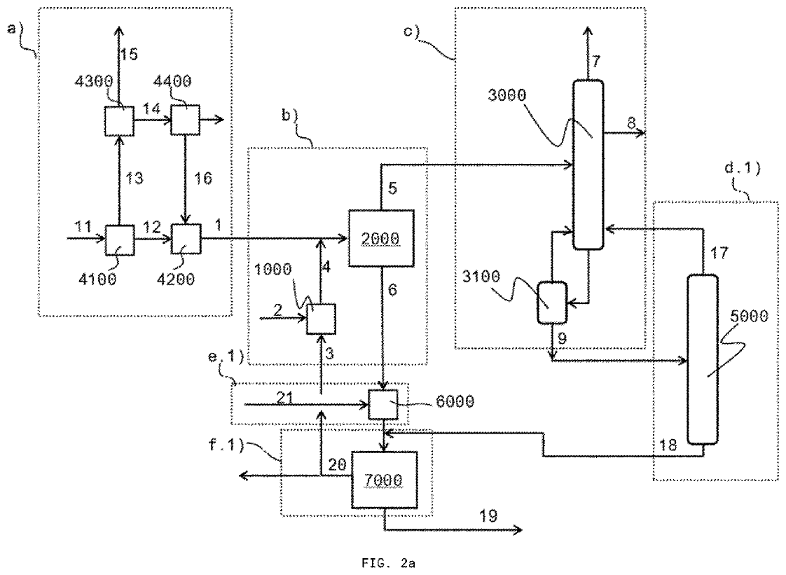

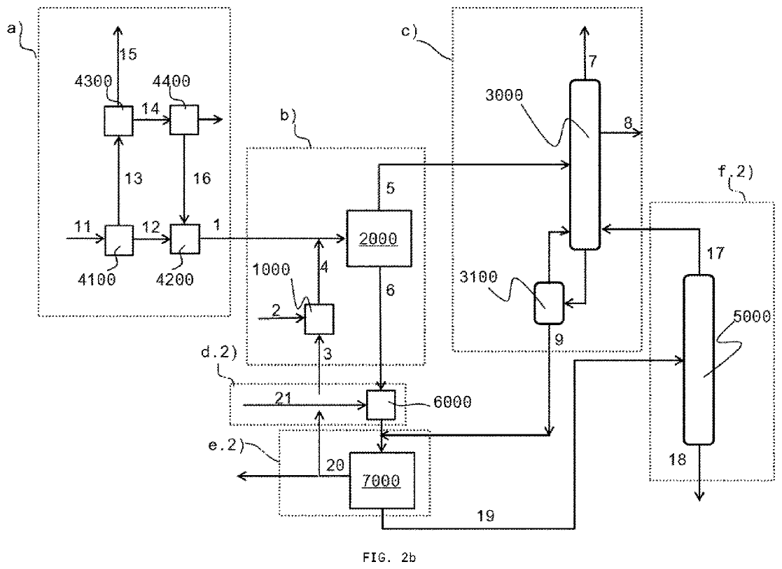

and preferably steps d) to f), comprising a stage of concentration, a stage of acid treatment and a stage of extraction, these stages existing in a first variant (steps d.1) to f.1); cf. also FIG. 2a) and a second variant (steps d.2) to f.2); cf. also FIG. 2b), which differ in the order of the stages and the manner in which the individual material streams are guided, wherein the first variant comprises:
d.1) concentrating the liquid stream (9) from step c) containing aniline and organic impurities having a higher boiling point than aniline by evaporation of aniline to obtain an aniline-containing distillate stream (17) and a bottom stream (18) containing organic impurities having a higher boiling point than aniline;
e.1) treating the aqueous aminophenolate phase (6) obtained in step b) with acid (21) to convert aminophenolate to aminophenol and (preferably in parallel with the addition of the acid)
f.1) extracting aminophenol formed by the acid treatment with an organic stream comprising the bottom stream (18) from step d.1) containing organic impurities having a higher boiling point than aniline, to obtain after phase separation an organic phase (19) and an aqueous phase (20), wherein alkali metal hydroxide is added to the aqueous phase and this aqueous phase is used as a constituent of the extractant (4) to be used in step a);

and wherein the second variant comprises:
d.2) treating the aqueous aminophenolate phase (6) obtained in step b) with acid (21) to convert aminophenolate to aminophenol and (preferably in parallel with the addition of the acid)
e.2) extracting aminophenol formed by the acid treatment with an organic stream comprising the liquid stream (9) from step c) containing aniline and organic impurities having a higher boiling point than aniline, to obtain after phase separation an organic phase (19) and an aqueous phase (20), wherein alkali metal hydroxide is added to the aqueous phase and this aqueous phase is used as a constituent of the extractant (4) to be used in step a);
f.2) concentrating the organic phase (19) from step e.2) by evaporation of aniline to obtain an aniline-containing distillate stream (17) and a bottom stream (18) containing organic impurities having a higher boiling point than aniline.

Both preferably conducted variants, the first and the second variant, can be combined with all other embodiments of the invention described hereinafter.

The process according to the invention using a mixture of an alkali metal hydroxide and an alkali metal salt different from an alkali metal hydroxide as extractant makes it possible, even in the case of comparatively low alkali metal hydroxide concentrations, to ensure smooth phase separation without (as for example taught in EP-A-1 845 080) having to resort to comparatively high extraction temperatures. The additional use of an alkali metal salt different from an alkali metal hydroxide in the extraction according to step b) makes it possible to reliably ensure that the aqueous phase during the phase separation is consistently the lower (“heavy”) phase, specifically without being limited in the choice of temperature and without increased use of the comparatively expensive alkali metal hydroxide. Furthermore, in a preferred configuration of the process according to the invention, it is possible after an acidification to back-extract the organic constituents from the aqueous phase, which makes it possible after replacing consumed base to recirculate essential constituents of the extractant. It is additionally possible, and is the subject matter of preferred embodiments, to supply the organic constituents removed in the back-extraction to a waste stream (obtained in any case), which makes disposal of additional waste streams superfluous.

In all industrially relevant processes for producing aniline, water is formed as coproduct (the water of reaction), meaning that a biphasic crude product composed of an aqueous phase (containing the water of reaction) and an organic phase (containing the aniline formed and organic impurities) is regularly initially obtained in the production of aniline. The aniline-containing phase remaining after partial to complete, preferably complete, removal of the aqueous phase from the crude product (by what is known to the person skilled in the art as a phase separation) is referred to in the context of the present invention as “crude aniline fraction”. As is known to the person skilled in the art, due to a certain residual solubility of the two phases in one another, such a separation of the aqueous phase from the organic phase of a biphasic process product is never perfect in the sense that a residue-free separation of organic constituents from aqueous constituents would be possible. The term “complete removal of the aqueous phase” or “complete phase separation” in the context of the present invention therefore only means that fractions of the aqueous phase are not intentionally left in the organic phase. Even with complete removal of the aqueous phase, the crude aniline fraction thus also comprises aqueous constituents alongside the organic constituents.

The term “aminopheno7” in the terminology of the present invention encompasses all isomers (thus the sum of all isomers present is meant), however, in this case experience has taught that only ortho- and para-aminophenol are regularly present in detectable amounts.

The mass concentrations of organic compounds specified in the context of the present invention relate, where these are measured values (and not values calculated from known feedstocks), to values determined by gas chromatography. The quantification of the concentration of low molecular weight organic compounds by means of gas chromatography is a method well-known to the person skilled in the art which requires no further explanation at this point. In the context of the present invention, the total mass of the organic constituents is a reference variable.

The ranges and preferred ranges specified above in step b) for the mass concentrations of alkali metal hydroxide on the one hand and of the alkali metal salt different from an alkali metal hydroxide on the other can in principle be combined with each other as desired, that is to say it is for example possible to use the alkali metal hydroxide in a concentration in the range from 0.010% by mass to 2.00% by mass (second-broadest range) and to use the alkali metal salt different from an alkali metal hydroxide in a concentration in the range from 12.0% by mass to 17.0% by mass (narrowest range). This also applies to the individual lower and upper limits. It is thus likewise possible, for example, to use the alkali metal hydroxide in a concentration in the range from 0.010% by mass to 2.00% by mass (second-broadest range) and to use the alkali metal salt different from an alkali metal hydroxide in a concentration in the range from 2.40% by mass (lower limit of the broadest range) to 17.0% by mass (upper limit of the narrowest range). However, particular preference is given to combining the mutually corresponding ranges with each other, that is to say for example the preferred range for the concentration of the alkali metal hydroxide with the preferred range for the concentration of the alkali metal salt different from an alkali metal hydroxide or the very preferred range for the concentration of the alkali metal hydroxide with the very preferred range for the concentration of the alkali metal salt different from an alkali metal hydroxide, and so on.

“Low boilers” in the context of the present invention are understood to be those organic compounds (and possibly azeotropes) having a lower boiling point than aniline. In terms of boiling behavior, water of course also belongs to the low boilers; however, because this is not an organic impurity attributable to side reactions and instead is either a coproduct of the aniline production (hydrogenation of nitrobenzene, ammonolysis of phenol) or is introduced with the reagents used (reduction of nitrobenzene with base metals such as iron in the presence of hydrochloric acid, ammonolysis with aqueous ammonia), it is considered separately in general and also in the context of the present invention. Typical low boilers in the context of the present invention are cyclohexylamine, cyclohexanone, cyclohexanol and benzene. Accordingly, “high boilers” in the context of the present invention are understood to be those organic compounds (and possibly azeotropes) having a higher boiling point than aniline. Typical high boilers are aminophenols, toluidines, phenylenediamine, diphenylamine, N-cyclohexylaniline or else unconverted starting material from the production, for example nitrobenzene. In principle, the classification of a substance as a low or high boiler is pressure-dependent, thus in the present case dependent on the pressure conditions selected for the distillation in step c) (a substance can be a low boiler at one particular pressure and a high boiler at another pressure). However, the examples mentioned above for typical low and high boilers correspond in all pressure ranges relevant for step c) to the assignment made. Phenol, however, has a boiling behavior that is so similar to that of aniline that it can be separated off as low or high boiler in step c) depending on the pressure present.

A “stream of purified aniline” is understood in the context of the present invention to be an aniline stream which has been subject to a distillative removal of low and high boilers. A stream of purified aniline obtained according to the invention contains, based on its total mass, aniline in a proportion in the range of 99.5000% by mass to 99.9999% by mass, preferably in the range from 99.9000% by mass to 99.9999% by mass, particularly preferably in the range from 99.9500% by mass to 99.9999% by mass.

In the appended drawings:

FIG. 1 shows an embodiment of the process according to the invention;

FIG. 2a shows a further embodiment of the process according to the invention comprising the first variant described above;

FIG. 2b shows a further embodiment of the process according to the invention comprising the second variant described above;

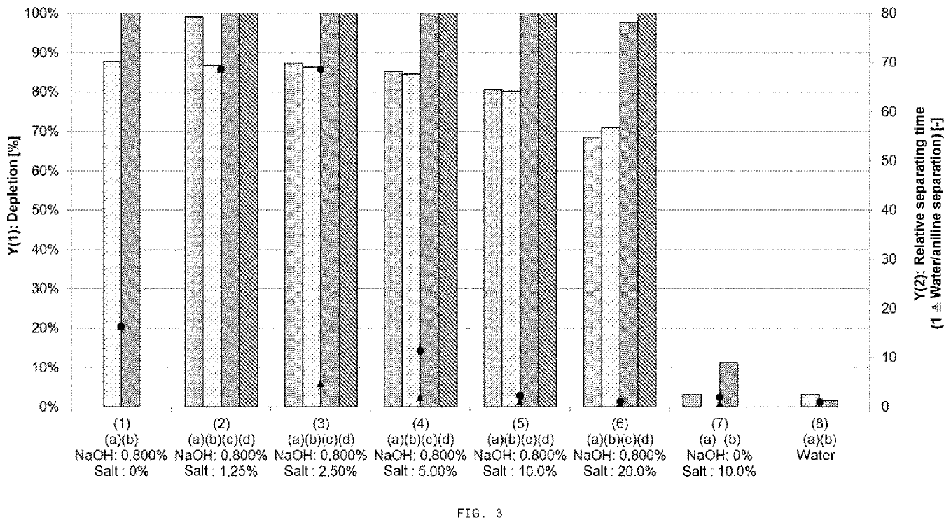

FIG. 3 shows the degree of depletion of phenol and aminophenol in an extraction of a phenol- and aminophenol-containing aniline using various extractants, and the relative phase separation time compared to extraction with pure water; and

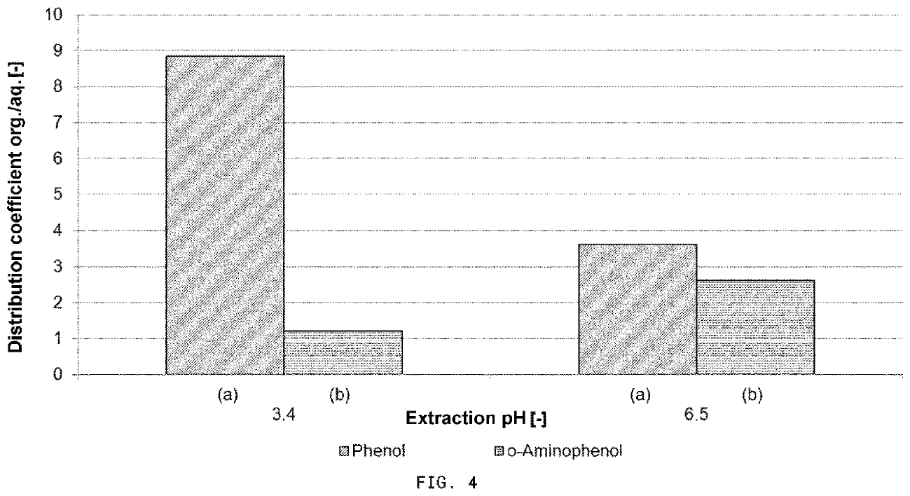

FIG. 4 shows the distribution coefficients of phenol and ortho-aminophenol between organic and aqueous phases in a biphasic mixture at different pH values.

What follows first is a brief summary of different possible embodiments of the invention, although the enumeration of the embodiments should be considered to be nonexhaustive.

In a first embodiment of the invention, which is combinable both with (see the statements above) the first variant and with the second variant, step c) comprises a distillation in a distillation column with side draw, wherein the stream of purified aniline is withdrawn as side stream, the gaseous stream containing organic impurities having a lower boiling point than aniline is withdrawn as top stream, and the liquid stream containing aniline and organic impurities having a higher boiling point than aniline is withdrawn as bottom stream from the distillation column with side draw.

In a second embodiment of the invention, which is combinable both with (see the statements above) the first variant and with the second variant, step c) comprises a distillation in two distillation columns connected in series, wherein in the first distillation column the gaseous stream containing organic impurities having a lower boiling point than aniline is withdrawn as top stream, wherein the bottom stream obtained in the first distillation column is conducted as feed into the second distillation column, wherein in the second distillation column the stream of purified aniline is obtained as top stream and the liquid stream containing aniline and organic impurities having a higher boiling point than aniline is obtained as bottom stream.

In a third embodiment of the invention, which is combinable both with (see the statements above) the first variant and with the second variant, step c) comprises a distillation in three distillation columns, wherein a top stream containing water and organic impurities is withdrawn from a first distillation column and is separated into an aqueous and an organic phase, the organic phase being conducted into a second distillation column from which the gaseous stream containing organic impurities having a lower boiling point than aniline is withdrawn as top stream, and wherein the bottom stream obtained in the first distillation column is conducted as feed into a third distillation column, wherein in the third distillation column the stream of purified aniline is obtained as top stream and the liquid stream containing aniline and organic impurities having a higher boiling point than aniline is obtained as bottom stream.

In a fourth embodiment of the invention, which is a particular configuration of the first embodiment to the third embodiment (that is to say, can be applied to each of these three embodiments), the gaseous stream containing organic impurities having a lower boiling point than aniline is partially liquefied, the liquid phase thus obtained, optionally after removal of any water present, is recycled (as reflux) into that distillation column from which the gaseous stream containing organic impurities having a lower boiling point than aniline was withdrawn as top stream, wherein the gaseous phase (containing, in addition to non-condensable gases, organic impurities having a lower boiling point than aniline) remaining after the partial liquefaction is discharged from the distillation.

In a fifth embodiment of the invention, which is combinable both with (see the statements above) the first variant and with the second variant, a distillation column is used for the concentration in step d.1) or in step f.2).

In a sixth embodiment of the invention, which is combinable both with (see the statements above) the first variant and with the second variant, the gaseous phase (containing, in addition to non-condensable gases, organic impurities having a lower boiling point than aniline) remaining in step c) after the partial liquefaction is condensed and the condensate obtained is used as an additional constituent of the organic stream to be used in step f.1) or step e.2).

In a seventh embodiment of the invention, which is combinable both with (see the statements above) the first variant and with the second variant,
the organic phase obtained in step f.1) after the phase separation or
the bottom stream obtained in step f.2) and containing organic impurities having a higher boiling point than aniline

is incinerated.

In an eighth embodiment of the invention, which is combinable both with (see the statements above) the first variant and with the second variant, the alkali metal salt used which is different from an alkali metal hydroxide is (i) an alkali metal chloride or (ii) an alkali metal sulfate.

In a ninth embodiment of the invention, which is a particular configuration of the eighth embodiment, the acid used in step e.1) or in step d.2) in case (i) is hydrochloric acid and in case (ii) is sulfuric acid.

In a tenth embodiment of the invention, which can be combined with all other embodiments, the extraction of the crude aniline fraction in step b) is conducted in several, preferably in two, steps in countercurrent flow.

In an eleventh embodiment of the invention, which can be combined with all other embodiments, the organic aniline phase obtained in step b) is mixed prior to the distillation in step c) with an aqueous solution of an alkali metal hydroxide and the mixture thus obtained is subject to the distillation of step c) without prior removal of the aqueous constituents thereof.

In a twelfth embodiment of the invention, which can be combined with all other embodiments, the crude aniline fraction is obtained by hydrogenation of nitrobenzene in the presence of a catalyst and removal of water formed in the hydrogenation.

In a thirteenth embodiment of the invention, which is a particular configuration of the twelfth embodiment, the hydrogenation of nitrobenzene is conducted in the gas phase and the gaseous reaction product obtained is condensed.

In a fourteenth embodiment of the invention, which is a particular configuration of the thirteenth embodiment, the condensation of the gaseous reaction product is conducted in two stages (namely a “partial condensation” and a “total condensation”) with successively falling temperature, wherein only after the second condensation stage a phase separation for removing water formed in the hydrogenation takes place.

In a fifteenth embodiment of the invention, which is a particular configuration of the fourteenth embodiment, the condensate obtained in the first condensation stage is combined with the organic phase remaining after the removal of water in the phase separation after the second condensation stage and is used (partially or completely) as crude aniline stream in step a).

In a sixteenth embodiment of the invention, which is a further particular configuration of the fourteenth embodiment, the crude aniline stream to be provided in step a) is withdrawn either from the condensate obtained in the first condensation stage or from the organic phase remaining after the removal of water in the phase separation after the second condensation stage.

In a seventeenth embodiment of the invention, which is a further particular configuration of the thirteenth embodiment, the condensation of the gaseous reaction product is conducted in one stage, wherein following the condensation a phase separation for removing water formed in the hydrogenation takes place, and wherein the organic phase remaining after removal of the water is used (partially or completely) as crude aniline stream in step a).

In an eighteenth embodiment of the invention, which is a particular configuration of the twelfth embodiment to the seventeenth embodiment, the hydrogenation is conducted adiabatically.

In a nineteenth embodiment of the invention, which is a further particular configuration of the twelfth embodiment to the seventeenth embodiment, the hydrogenation is conducted isothermally.

In a twentieth embodiment of the invention, which is a particular configuration of the eighteenth embodiment, the catalyst comprises copper on a silicon dioxide support.

In a twenty-first embodiment of the invention, which is a particular configuration of the nineteenth embodiment, the catalyst comprises palladium on an aluminum oxide support.

In a twenty-second embodiment of the invention, which can be combined with all other embodiments, the alkali metal of the alkali metal hydroxide and of the alkali metal salt different from an alkali metal hydroxide is in each case sodium or potassium.

In a twenty-third embodiment of the invention, which can be combined with all other embodiments, in step b) a molar ratio of alkali metal hydroxide to phenolic hydroxyl groups is set in the range from 0.80 to 50, preferably in the range from 0.95 to 10, particularly preferably in the range from 1.0 to 5.0, and very particularly preferably in the range from 1.0 to 2.4.

In a twenty-fourth embodiment of the invention, which can be combined with all other embodiments, the extraction in step b) is conducted at a temperature in the range from 20° C. to 95° C., particularly preferably 25° C. to 85° C., very particularly preferably 25° C. to 70° C. and extremely particularly preferably 25° C. to 40° C.

In a twenty-fifth embodiment of the invention, which can be combined with all other embodiments comprising steps d) to f) (in the first or second variant), the extraction of aminophenol formed by the acid treatment with an organic stream (step f.1 or step e.2) is conducted in a mixer-settler apparatus (mixer-settler) which comprises a mixing unit and a separating unit, wherein the treatment of the aqueous aminophenolate phase (6) obtained in step b) with acid (21) to convert aminophenolate to aminophenol is conducted such that the aqueous aminophenolate phase (6) obtained in step b) is mixed in the mixing unit with the acid (21) and the organic stream (comprising stream 18 or 9) (simultaneous addition of the acid and the organic stream to the aqueous aminophenolate phase, that is to say the extraction with the organic stream is conducted in parallel with the acid treatment), and this is followed by a phase separation into an organic phase (19) and an aqueous phase (20) in the separating unit.

In a twenty-sixth embodiment of the invention, which can be combined with all other embodiments,

the alkali metal hydroxide used is
sodium or potassium hydroxide in a concentration range from 0.009% by mass to 2.05% by mass, preferably 0.010% by mass to 2.00% by mass, particularly preferably 0.010% by mass to 1.00% by mass, very particularly preferably 0.50% by mass to 1.00% by mass, extremely particularly preferably 0.76% by mass to 1.00% by mass,

and the alkali metal salt used which is different from an alkali metal hydroxide is
either
sodium or potassium chloride in a concentration range from 4.00% by mass to 25.0% by mass, preferably 5.00% by mass to 25.0% by mass, particularly preferably 10.0% by mass to 20.0% by mass, very particularly preferably 12.0% by mass to 17.0% by mass,
or
sodium or potassium sulfate in a concentration range from 2.40% by mass to 25.0% by mass, preferably 4.00% by mass to 25.0% by mass, particularly 5.00% by mass to 25.0% by mass, very particularly preferably 10.0% by mass to 20.0% by mass, extremely particularly preferably 12.0% by mass to 17.0% by mass.

The embodiments briefly outlined above and further possible embodiments of the invention are elucidated in more detail hereinafter. Embodiments may be combined with one another as desired, unless the opposite is apparent from the context.

FIG. 1 shows a simple configuration of the process according to the invention. The reference symbols have the following meanings.
Apparatus or process steps:
   1000 mixer;
   2000 base extraction including phase separation;
   3000 distillation column;
   3100 bottom evaporator of the distillation column 3000.
Material streams:
   1 crude aniline fraction;
   2 alkali metal hydroxide solution;
   3 alkali metal salt solution different therefrom (preferably alkali metal chloride solution or alkali metal sulfate solution);
   4 aqueous extractant;
   5 organic aniline phase depleted in aminophenol (and phenol);
   6 aqueous aminophenolate phase;
   7 top stream of the distillation column (low boilers, water, non-condensable gases);
   8 purified aniline;
   9 discharge from the bottom evaporator;
   10 distillate from the bottom evaporator.

The industrial processes currently customary in the prior art for the production of aniline (especially the hydrogenation of nitrobenzene, to a lesser extent the reduction of nitrobenzene with base metals such as iron, and—nowadays of only minor significance—the ammonolysis of phenol or chlorobenzene) despite their differences all ultimately yield a phenol- and aminophenol-containing crude aniline, so that the process according to the invention can be reasonably applied to all of these production processes and hence the crude aniline to be provided in step a) can originate from all of these processes as well.

The actual production of the aniline to be purified is preferably effected by the catalytic hydrogenation of nitrobenzene. The crude process product obtained in this case contains, in addition to aniline and secondary components, considerable proportions of water as coproduct of the hydrogenation (water of reaction), which is partially to completely, preferably completely, removed prior to the further purification. The removal is effected by phase separation known to the person skilled in the art. The aqueous phase obtained thereby in the preferred configuration with complete phase separation (see the explanations above in this respect) contains the major portion of the water of reaction (except for a residual amount which cannot be removed by phase separation due to a low solubility of water in aniline) and is removed. In addition to aniline, the remaining organic phase also contains organic impurities and dissolved water.

The hydrogenation can advantageously be conducted in the gas phase, with hydrogen preferably being used in stoichiometric excess. As is known to the person skilled in the art, the hydrogenation can be conducted adiabatically (without controlled removal of the heat of reaction) or isothermally (with controlled removal of the heat of reaction, for example in a shell and tube reactor using a cooling heat transfer medium such as water, an oil or a salt melt).

In the case of the adiabatic mode, two or more reactors are generally arranged successively in series. Here, the heat of reaction leads to a considerable increase in the temperature of the reaction gas (what is known as an “adiabatic temperature jump”), so that the latter is cooled after exiting a reactor. After cooling, gaseous nitrobenzene and further hydrogen can optionally be added anew and supplied to a further reactor. To dilute the heat of reaction, adiabatic processes generally operate with particularly large excesses of hydrogen. Unconsumed hydrogen is therefore preferably circulated.

In the isothermal process, a single reactor is generally used. This is actively cooled as described. Despite the generally much smaller excesses of hydrogen compared to the adiabatic process, circulation of unconsumed hydrogen is usually expedient.

The advantages of the adiabatically conducted process reside in the simpler design of the reactors, since no cooling of the catalyst bed is required.

The isothermal process is correspondingly more expensive due to the necessary cooling of the catalyst bed. However, due to the achievable reactor sizes, no series arrangement of two or more reactors and the corresponding intercoolers is required in general here.

The process according to the invention is in particular advantageously usable when the hydrogenation is conducted isothermally using a catalyst containing palladium on an aluminum oxide support or adiabatically using a catalyst containing copper on a silicon dioxide support. In both cases, in addition to phenol, relatively large amounts of aminophenols are formed, which can be removed in an efficient manner by the process according to the invention.

The gaseous reaction product initially obtained in both procedures is condensed. Such a condensation can take place in one stage (i.e. to obtain a single condensate), followed by a phase separation. The organic phase remaining after removal of the water of reaction can be supplied to step b) as crude aniline fraction.

However, it is also possible to conduct the condensation in two stages such that two condensates of different compositions are obtained. To this end, the gaseous crude product stream initially obtained is exposed to successively falling temperatures so that at first only the higher boiling components condense out together with aniline. In this way, a first condensate (the so-called “partial condensate”) is obtained which contains hardly any water and preferably is monophasic, so that a phase separation is in this case superfluous. This “partial condensate” thus essentially consists of aniline and high boilers. It is only in the second condensation conducted at lower temperature (the so-called “total condensation”) that low boilers and water in addition to further aniline also condense out. The phase separation for removing the water of reaction can therefore remain restricted to this “total condensate”. The organic phase remaining after the phase separation can advantageously be combined with the partial condensate and supplied to step b) as crude aniline fraction. However, it is also possible to supply only one of the two streams—partial condensate or the organic phase of the total condensate, obtained after phase separation—as crude aniline fraction to step b).

Irrespective of the exact manner in which the crude aniline is provided, the present invention can be used for the purification of crude aniline fractions having a concentration of aminophenol, based on the total mass of the organic constituents of the crude aniline fraction, in the range from 0.001% by mass to 1.00% by mass, preferably in the range from 0.001% by mass to 0.50% by mass, particularly preferably in the range from 0.001% by mass to 0.10% by mass. Since the formation of aminophenol is always accompanied by the formation of phenol, such crude aniline fractions regularly also contain phenol, specifically in particular in the range from 0.001% by mass to 1.00% by mass, preferably in the range from 0.001% by mass to 0.50% by mass, particularly preferably in the range from 0.001% by mass to 0.10% by mass, based on the total mass of the organic constituents of the crude aniline fraction.

As long as the requirement according to the invention of the additional use of an alkali metal salt different from an alkali metal hydroxide is observed, the extraction in step b) can in principle be conducted using apparatuses and procedures known per se in the prior art. Without wishing to be tied to a theory, it is assumed that the alkali metal salt different from an alkali metal hydroxide ensures a sufficient density difference between the aqueous and the organic phase even in the event of comparatively low alkali metal hydroxide concentrations in the extractant and hence promotes simple phase separation. Such promotion of the separation of two immiscible or very poorly miscible phases should be distinguished from the procedure known in organic chemistry of lowering the solubility of organic compounds in an aqueous phase by saturating the latter with salt and hence of raising the yield of organic target product in the workup of aqueous-organic process products (by minimizing the loss via the residual solubility of this organic target product in the aqueous phases).

The two constituents of the aqueous extractant that are essential to the invention, the alkali metal hydroxide (the actual reactive constituent) and the alkali metal salt different from an alkali metal hydroxide, can be supplied to the extraction in premixed form (which is preferred) or else separately (via separate supply devices). The extraction can be conducted in one or more stages in apparatuses known to the person skilled in the art, in particular in so-called “mixer-settler” apparatuses (mixer-settlers). Particular preference is given to a multistage, especially two-stage, extraction in countercurrent flow. The phases are separated after each extraction step and the organic phase obtained after the final (possibly only) extraction step, which is depleted in aminophenol (and as a matter of course also in phenol), is supplied to step c).

The extraction in step b) is preferably conducted at a temperature in the range from 20° C. to 95° C., particularly preferably 25° C. to 85° C., very particularly preferably 25° C. to 70° C. and extremely particularly preferably 25° C. to 40° C.

According to the invention, based on the total mass of the extractant, the alkali metal hydroxide is used in a concentration range from 0.009% by mass to 2.05% by mass, preferably 0.010% by mass to 2.00% by mass, particularly preferably in the concentration range from 0.010% by mass to 1.00% by mass, very particularly preferably in the concentration range from 0.50% by mass to 1.00% by mass, extremely particularly preferably in the concentration range from 0.76% by mass to 1.00% by mass, and the alkali metal salt different from an alkali metal hydroxide is used in a concentration range from 2.40% by mass to 25.0% by mass, preferably 4.00% by mass to 25.0% by mass, particularly preferably in the concentration range from 5.00% by mass to 25.0% by mass, very particularly preferably in the concentration range from 10.0% by mass to 20.0% by mass, extremely particularly preferably in the concentration range from 12.0% by mass to 17.0% by mass. As already mentioned, preference is given to premixing the two constituents essential to the invention. In the event of the likewise possible separate supply of the two constituents essential to the invention into the extraction apparatus used for step b) (in which the extractant first forms in the extraction apparatus as a result of mixing), the reference variable for the mass concentrations mentioned is the sum of the individual masses of the two constituents essential to the invention (which is equal to the mass of the premixed extractant in the preferred embodiment).

In a preferred embodiment, the total concentration of phenol and aminophenols in the crude aniline fraction is monitored continuously or at intervals and the composition and/or the amount of the extractant is matched thereto. For a maximally complete removal of aminophenol (and phenol), the alkali metal hydroxide must be used in at least a stoichiometric amount, preferably in excess, based on the phenolic hydroxyl groups. (Phenolic hydroxyl groups in the context of the present invention collectively refers to the hydroxyl groups of phenol and aminophenols; phenol and aminophenols are also referred to collectively as phenolic compounds). Under certain circumstances, however, it may also be expedient to deliberately not neutralize all phenolic compounds in step b) and instead to shift part of the removal into the following distillation of step c). If the distillation in step c) can remove a certain amount of phenolic compounds without problem, the use of the extractant in step b) can in this way be minimized. The relative amount of alkali metal hydroxide to be advantageously used can therefore vary depending on the exact procedure. In particular, useful molar ratios of alkali metal hydroxide to phenolic hydroxyl groups have proven to be those in the range from 0.80 to 50, preferably in the range from 0.95 to 10, particularly preferably in the range from 1.0 to 5.0 and very particularly preferably in the range from 1.0 to 2.4. In the context of the present invention, the molar ratios can be directed solely toward the aim of reducing the concentration of phenolic hydroxyl groups under the present conditions to the extent necessary. Moreover, it is in particular not necessary to add further alkali metal hydroxide to establish a desired density as may absolutely be required in the prior art processes. In contrast, possibly required adjustments of the density in the process according to the invention can be made in a simple and cost-effective manner by addition of the alkali metal salt different from an alkali metal hydroxide.

It may also be expedient to add further aqueous alkali metal hydroxide to the aniline phase obtained in the extraction and depleted in aminophenol prior to the distillation of said phase in step c) and to subject the mixture thus obtained to the distillation of step c) without removal of its aqueous constituents. This is equivalent to a combination of the basic extraction according to the invention in the presence of an alkali metal salt different from an alkali metal hydroxide and the distillation in the presence of base known from the literature.

The alkali metal hydroxide used is preferably sodium or potassium hydroxide. The alkali metal salt used which is different from an alkali metal hydroxide is preferably sodium or potassium chloride or sodium or potassium sulfate. The alkali metal salt of the hydroxide used should preferably be the same as that of the alkali metal salt used which is different from an alkali metal hydroxide, in particular in each case sodium or potassium.

In step c) the distillative (fine) purification of the crude aniline prepurified by the extraction in step b) takes place. In principle, all distillation apparatuses and methods known to the person skilled in the art as suitable for aniline purification can be used.

In a first configuration option for step c) of the present invention, the low boilers and dissolved water on the one hand and the high boilers on the other are removed in separate distillation columns, that is to say that the distillative purification comprises two distillation columns connected in series. The feed of the first distillation column is the crude aniline fraction prepurified by basic extraction from step b).

In the first distillation column (the so-called “low boiler column”), low boilers are distilled off overhead whereas aniline and high boilers accumulate in the bottoms. Dissolved water can be drawn off likewise overhead or else in a side draw. The bottoms of the first column forms the feed of a further (the second) distillation column (the so-called “aniline column”), in which aniline is distilled off overhead and in the bottoms of which an aniline-containing high boiler stream accumulates. The aniline distilled off overhead in this embodiment constitutes the stream of purified aniline for the purposes of the invention.

In the first configuration option, the “low boiler distillation” can preferably be conducted at an overhead pressure in the range from 1000 mbar_{(abs.)} to 1100 mbar_{(abs.)} and an overhead temperature in the range from 97° C. to 100° C. The distillation in the aniline column is preferably conducted at an overhead pressure in the range from 100 mbar_{(abs.)} to 200 mbar_{(abs.)} and an overhead temperature in the range from 110° C. to 130° C.

In a second configuration option for step c) of the present invention, dissolved water is removed in a dedicated distillation column (the so-called “dewatering column”). The crude aniline fraction prepurified by basic extraction from step b) is supplied to this dewatering column (in this configuration option the first distillation column). A stream containing water, aniline and low boilers is withdrawn at the top of the dewatering column and is then separated into an aqueous and an organic phase. The organic phase is fed to a further distillation column (the “low boiler column”, in this configuration option the second distillation column), in which low boilers are distilled off overhead. The bottom stream of this low boiler column is recycled to the dewatering column.

The bottoms of the dewatering column forms the feed of a third distillation column (the so-called “aniline column”), in which, as in the first configuration option, aniline is distilled off overhead and in the bottoms of which an aniline-containing high boiler stream accumulates. The aniline distilled off overhead constitutes, as in the first configuration option, the stream of purified aniline for the purposes of the invention.

In the second configuration option, the “dewatering column” can preferably be operated at an overhead pressure in the range from 100 mbar_{(abs.)} to 400 mbar_{(abs.)} and an overhead temperature in the range from 100° C. to 150° C. The “low boiler distillation” is preferably conducted at an overhead pressure in the range from 1000 mbar_{(abs.)} to 1100 mbar_{(abs.)} and an overhead temperature in the range from 70° C. to 90° C. The distillation in the aniline column is preferably conducted at an overhead pressure in the range from 250 mbar_{(abs.)} to 350 mbar_{(abs.)} and an overhead temperature in the range from 140° C. to 160° C.

However, it is also possible, and is the subject of a third configuration option for step c) of the present invention, for the crude aniline fraction prepurified by basic extraction from step b) to be introduced into a distillation column with side draw (also referred to as side draw column), in which the low and high boilers (and also dissolved water) are removed in a single distillation step. In one embodiment of the third configuration option, the side draw column has a dividing wall (what is known as a dividing wall column). In the third configuration option, the stream of purified aniline is withdrawn from the distillation column as side draw, while low boilers (and water) are drawn off overhead. As in the aniline column of the configuration options mentioned above, an aniline-containing high boiler stream accumulates in the bottoms. This distillation is preferably conducted at an overhead pressure of the side draw column in the range from 200 mbar_{(abs.)} to 500 mbar_{(abs.)} and at an overhead temperature of the side draw column in the range from 100° C. to 150° C.

In all cases, the low boiler stream (that is to say in the first configuration option the top stream of the first distillation column, in the second configuration option the top stream of the second distillation column and in the third configuration option the top stream of the side draw column) is initially obtained in gas form. This gaseous low boiler stream is preferably partially liquefied, wherein the liquid phase thus obtained, optionally after removal of any water present, can advantageously be sent back to the initial column as reflux. The uncondensed gas phase, which, in addition to a proportion of non-condensable (under conditions customary in industry) gases such as for example nitrogen, essentially consists of low boilers, is discharged from the distillation.

It may be advantageous to subject the aniline-containing high boiler stream obtained in the mentioned configuration options for step c) to a concentration of the high boilers in a step d.1) in order in this way to minimize the loss of the product of value aniline (corresponding to the abovementioned first variant). In the process, an aniline-containing stream is evaporated and can advantageously be recycled into the distillation of step c). This aniline-containing stream is preferably led into the bottom of that distillation column from which the aniline-containing high boiler stream originally came from. In addition, a bottom stream (high boiler stream) containing organic impurities having a higher boiling point than aniline is obtained in this step. The concentration of step d.1) is preferably effected in a distillation column provided specifically for this purpose.

The economics of the process according to the invention can be further improved in the first variant if the aqueous aminophenolate phase obtained in the extraction step, step b), is treated with acid in a step e.1) to convert aminophenolate to aminophenol and is extracted with an organic stream in a step f.1), to obtain after phase separation an organic phase (enriched in aminophenol) and an aqueous phase (depleted in aminophenol). It is preferable to conduct step f.1) in parallel with step e.1) (=simultaneous addition of acid and organic stream to the aqueous aminophenolate phase) in order to prevent aminophenols precipitating out as solids. Alkali metal hydroxide can then advantageously be added to this aqueous phase and this aqueous phase can be used as a constituent of the extractant to be used in step a). It is possible to supply the entire amount of the alkali metal hydroxide to be used in step a) in this way. It is also possible to supply the entire amount of the alkali metal salt to be used in step a) and different from an alkali metal hydroxide in this way, that is to say in the form of the salt of the acid used in step e.1), this salt being present in the aqueous phase (depleted in aminophenol) in this embodiment in any case. A particularly suitable organic stream for the extraction is the high boiler bottom stream obtained in step d.1). This high boiler bottom stream can also be mixed with other organic streams for this purpose. For example, the uncondensed gas phase obtained in step c) during the preferably conducted partial liquefaction of the gaseous low boiler top stream, which, in addition to a proportion of non-condensable (under conditions customary in industry) gases such as for example nitrogen, essentially consists of low boilers, can be converted into a liquid low boiler stream by liquefaction of its condensable fractions, said liquid low boiler stream being suitable for mixing with the high boiler bottom stream obtained in step d.1) for the purpose of extracting aminophenol. In any case, the aniline content of the aminophenol-containing organic phase obtained after extraction and phase separation is so low that this organic phase can be incinerated without appreciable economic losses. In step f.1), aminophenols (and of course also phenols, where present) are transferred back from an aqueous phase into an organic phase (they do indeed originally come from an organic phase, the crude aniline fraction); thus this step is also referred to as a back-extraction.

The second variant described above also makes use of a back-extraction in this sense (here step e.2)). However, in contrast to the first variant, the organic stream here is the liquid stream from step c) containing aniline and organic impurities having a higher boiling point than aniline, while the acid treatment (here step d.2)) can in principle be conducted as in the first variant. In contrast to the first variant, here the organic phase obtained in the back-extraction is concentrated (in step f.2)), wherein this step is configured such that a reintroduction into step c) of aminophenols/phenols via the distillate of step f.2) is prevented. However, due to the comparatively small size of the stream of organic phase which is supplied to the concentration, this is possible without endangering the energy-related advantages of the process. The advantage of this second variant can be seen to be a more robust process regime which offers greater degrees of freedom. The amount of the organic stream which is sent to the back-extraction—here stream 9—can in the second variant if required be chosen to be greater than in the first variant (the amount of said organic stream—stream 18—there is essentially determined by the concentration of high boilers in the crude aniline fraction). Aniline contained in stream 9 passes into stream 19 and is recycled into the distillation column for concentrating the high boilers (5000), from where it passes via stream 17 into the aniline distillation (3000) and therefore is not lost. In the second variant, the amount of the organic stream—here the liquid stream from step c) containing aniline and organic impurities having a higher boiling point than aniline (stream 9)—can therefore be chosen independently of the amount of high boilers being obtained. Thus a phase separation on a scale relevant for industrial applications can be conducted in e.2) for all compositions of the crude aniline fraction. In the second variant, too, it is preferable to conduct the step of extraction with the organic stream (here step e.2)) in parallel with the step of acid treatment (here step d.2)) (=simultaneous addition of acid and organic stream to the aqueous aminophenolate phase) in order to prevent aminophenols precipitating out as solids.

A suitable acid for the acid treatment of step e.1) or step d.2) is in particular hydrochloric acid or sulfuric acid. The acid is chosen corresponding to the choice of the alkali metal salt used in step a) which is different from an alkali metal hydroxide; in the case of an alkali metal chloride (especially sodium or potassium chloride) hydrochloric acid is used and in the case of an alkali metal sulfate (especially sodium or potassium sulfate) sulfuric acid is used. The acid treatment in step e.1) or step d.2) is preferably effected to a pH (20° C.) in the range from 2.0 to 8.5, preferably to a pH (20° C.) in the range from 3.5 to 8.5, particularly preferably to a pH (20° C.) in the range from 5.0 to 8.5.

FIG. 2a shows a preferred configuration of the process according to the invention in the first variant with two-stage condensation of the crude product of a gas-phase aniline hydrogenation; concentration of the bottom stream of the aniline distillation and extraction of the waste water, obtained in the base extraction, with the high boiler stream from the concentration step. FIG. 2b shows a corresponding configuration for the second variant. Reference symbols identical to those in FIG. 1 each have the same meaning as in that figure. The further reference symbols have the following meanings.

Apparatus or process steps:
4100 partial condensation;
4200 mixer;
4300 total condensation;
4400 phase separation;
5000 distillation column for concentration of the high boilers;
6000 neutralization;
7000 extraction including phase separation.

Material streams:
11 gaseous crude product of the aniline hydrogenation;
12 partial condensate;
13 fractions of the crude product which have not condensed in the
partial condensation;
14 total condensate;
15 fractions which have not condensed in the total condensation
(essentially excess hydrogen);
16 organic phase of the total condensate;
17 aniline-containing distillate stream;
18 bottom stream containing high boilers (in the second variant according to FIG. 2b: preferably prior to incineration);
19 high boiler stream from extraction (in the first variant according to FIG. 2a: preferably prior to incineration);
20 aqueous phase (depleted in aminophenol) (added to stream 3; a portion may also be discharged as waste water);
21 acid (preferably hydrochloric or sulfuric acid).

The representation of the neutralization and extraction (including the phase separation) in FIG. 2a/b in two stages 6000 and 7000 does not mean that these process steps necessarily have to be conducted in two apparatuses. In a preferred embodiment, the extraction is conducted, as already mentioned, in what is known as a “mixer-settler” apparatus, that is to say an apparatus which includes a mixing unit and a separating unit. In a particularly preferred configuration of this embodiment the acid (stream 21 in the figures) and the organic stream (stream 18 and stream 9 in figures FIG. 2a and FIG. 2b, respectively) are fed into the mixing unit and this is followed by a phase separation in the separating unit.

### EXAMPLES

### Example 1

### Proof-of-Principle Experiments for Depletion of Aminophenol and for Phase Separation Time (see FIG. 3)

The crude aniline for purification contained, based on the total mass of its organic constituents, 750 ppm of phenol and 500 ppm of aminophenols, and was saturated with water. This mixture was extracted in various experiments with water, sodium hydroxide solution, sodium chloride solution, and solutions containing sodium hydroxide and sodium chloride, a volume ratio of aniline phase to extractant of 4:1 being observed. The NaOH concentration was in this case adjusted to a constant value of 0.800% by mass with various NaCl concentrations (0% by mass, 1.25% by mass, 2.50% by mass, 5.00% by mass, 10.0% by mass and 20.0% by mass). The series of experiments was also conducted in analogous fashion with sodium sulfate solutions instead of NaCl solutions.

It was found that in the experiment with a salt concentration of 5.00% by mass the phase separation was considerably accelerated in the case of both salts, NaCl and Na₂SO₄. In the experiment with a salt concentration of 10.0% by mass, the time needed for the phase separation in the case of both salts was comparable to that of the pure water-aniline mixture. At a salt concentration of 2.50% by mass, a considerable shortening of the phase separation time was also observed in the case of sodium sulfate.

In all tests (except for when using pure water and pure sodium chloride solution), ortho-aminophenol was removed from the organic phase to below the limit of detection, the added phenol could be removed from the crude aniline to an extent of 75% to 85%. No formation of solids was observed in any of the tests.

The results are presented in FIG. 3 in the form of a diagram.

The percentage depletion of phenol/ortho-aminophenol is presented on the left-hand ordinate axis (Y1) relating to the height of the bars (for example, a value of 100 on the left-hand ordinate axis means that the corresponding phenolic compound has been depleted to below the limit of detection).

The relative separating time (time requirement for the phase separation) is presented on the right-hand ordinate axis (Y2), relating to the position of the triangles (separating time for experiments using sodium sulfate) and of the circles (experiments using sodium chloride and experiments without the addition of a further salt). For this purpose, the time requirement for the separation of aniline of the abovementioned composition of demineralized water was set to 1 (=reference), and the remaining values were reported in relation to this (for example, a value of 80 on the right-hand ordinate axis means that the phase separation lasted 80 times as long as in the reference case).

The result of every series of experiments is presented on the abscissa axis in the form of a plurality of bars and symbols (triangles, circles) which have the meanings given above. The following series of experiments are shown from left to right in this figure.

Extraction of the crude aniline with:
(1) sodium hydroxide solution without further salt content,
(2) sodium hydroxide solution containing a proportion by mass of further salt of 1.25%,
(3) sodium hydroxide solution containing a proportion by mass of further salt of 2.50%,
(4) sodium hydroxide solution containing a proportion by mass of further salt of 5.00%,
(5) sodium hydroxide solution containing a proportion by mass of further salt of 10.0%,
(6) sodium hydroxide solution containing a proportion by mass of further salt of 20.0%,
(7) water (without NaOH) containing a proportion by mass of salt of 10.0% and
(8) demineralized water (containing neither NaOH nor further salt).

In experiment series (1) the bars denote, from left to right, the percentage depletion of:
(a) phenol and
(b) ortho-aminophenol,

in each case without the addition of a further salt.

In experiment series (2) to (8) the bars denote, from left to right, the percentage depletion of:
(a) phenol when using sodium sulfate as further salt,
(b) phenol when using sodium chloride as further salt,
(c) ortho-aminophenol when using sodium sulfate as further salt, and
(d) ortho-aminophenol when using sodium chloride as further salt.

In experiment series (7) the bars denote, from left to right, the percentage depletion of:
(a) phenol when using sodium sulfate as further salt, and
(b) ortho-aminophenol when using sodium sulfate as further salt.

In experiment series (8) the bars denote, from left to right, the percentage depletion of:
(a) phenol and
(b) ortho-aminophenol,
in each case without the addition of a further salt.

### Example 2

### Proof-of-Principle Experiments for Neutralization of the Aqueous Phase Depleted in Aminophenol and Subsequent Extraction of the Aminophenol Formed (see FIG. 4)

An aqueous NaOH solution (concentration 0.800% by mass) was saturated with aniline and then doped with 5000 ppm of ortho-aminophenol and 5000 ppm of phenol, so that the salts of the respective phenolic compounds were present. Various sample(s) were then adjusted with hydrochloric acid of a concentration of 30% by mass to a pH in the range from 6.0 to 7.0 and below 3.5. The acidified samples were then extracted with a synthetic high boiler waste stream consisting of diphenylamine (as exemplary high boiler) and aniline in the ratio by mass of 1:1. It was possible here to reduce the concentration of all phenolic compounds in the aqueous medium by a factor of 1.5 to 9.0, depending on the pH and the nature of the phenolic compound. It can also be seen that the phenolic compounds can be extracted with varying degrees of success depending on the pH value.

The results are presented in FIG. 4 in the form of a bar chart.

The distribution coefficient of the respective phenolic compound as the ratio of the proportion by mass of the respective phenolic compound in the organic phase to the proportion by mass thereof in the aqueous phase is reported on the ordinate axis (Y), relating to the height of the bars.

The result of each series of experiments is shown on the abscissa axis (X) each in the form of two bars at the pH values of 3.4 and 6.5. In this case, each left-hand bar (a) indicates the distribution coefficient of phenol and each right-hand bar (b) indicates the distribution coefficient of ortho-aminophenol.

### Example 3

### Continuous Experiments with Respect to the Process

Production samples of a crude aniline (containing approx. 2500 ppm of phenol and 250 ppm of aminophenol) were continuously contacted in a two-stage mixer-settler system with an aqueous solution containing 10% by mass of Na₂SO₄ and 1% by mass of NaOH (pH=12) in a volume ratio of 4:1 (ratio of organic phase to aqueous phase) (extraction). An organic raffinate stream and an aqueous extract stream were obtained. The aqueous extract stream was continuously adjusted to a neutral pH by addition of sulfuric acid and contacted in a two-stage mixer-settler with a synthetically produced high boiler stream (containing 50% by mass of aniline and 50% by mass of diphenylamine as exemplary representatives of typical high boilers of an aniline process) in a volume ratio of 2:1 (ratio of aqueous phase to organic phase) (acid treatment and back-extraction). Extraction and back-extraction were conducted continuously and in series at a temperature of 30° C. It was found that phenol and aminophenol were no longer detectable in the aniline raffinate from the extraction. This confirms the proof-of-principle experiments for the case of a continuous process regime as well. During the back-extraction, phenol and aminophenol were found at high concentrations in the high boiler extract. Considered over the whole workup process, it was found that on average 85% of the phenol and 95% of the aminophenol could be transferred from the crude aniline stream into the back-extraction stream (in the high boiler extract). The phase separation proceeded without problems in all separators, the addition of the salt Na₂SO₄ resulting in the aqueous phase being obtained as the heavy phase in all separators.

1. A process for the purification of aniline, comprising:
a) providing a crude aniline fraction containing aniline and organic impurities, wherein the organic impurities comprise phenol, organic impurities having a lower boiling point than aniline, and aminophenol, and also further organic impurities having a higher boiling point than aniline, wherein the concentration of aminophenol, based on the total mass of the organic constituents of the crude aniline fraction, is in the range from 0.001% by mass to 1.00% by mass;
b) extracting the crude aniline fraction from step a) with an aqueous extractant containing, based on the total mass of the aqueous extractant, an alkali metal hydroxide in a concentration range from 0.009% by mass to 2.05% by mass and an alkali metal salt different from an alkali metal hydroxide in a concentration range from 2.40% by mass to 25.0% by mass,
wherein, after phase separation, an organic aniline phase depleted in aminophenol and an aqueous aminophenolate phase are obtained; and
c) distilling the organic aniline phase from step b) to obtain a stream of purified aniline, a gaseous stream containing organic impurities having a lower boiling point than aniline, and a liquid stream containing aniline and organic impurities having a higher boiling point than aniline.

2. The process as claimed in claim 1, further comprising:
d.1) concentrating the liquid stream from step c) containing aniline and organic impurities having a higher boiling point than aniline by evaporation of aniline to obtain an aniline-containing distillate stream and a bottom stream containing organic impurities having a higher boiling point than aniline;
e.1) treating the aqueous aminophenolate phase obtained in step b) with acid to convert aminophenolate to aminophenol; and
f.1) extracting aminophenol formed by the acid treatment with an organic stream comprising the bottom stream from step d.1) containing organic impurities having a higher boiling point than aniline, to obtain, after phase separation, an organic phase and an aqueous phase, wherein alkali metal hydroxide is added to the aqueous phase and this aqueous phase is used as a constituent of the extractant used in step a).

3. The process as claimed in claim 1, further comprising:
d.2) treating the aqueous aminophenolate phase obtained in step b) with acid to convert aminophenolate to aminophenol;
e.2) extracting aminophenol formed by the acid treatment with an organic stream comprising the liquid stream from step c) containing aniline and organic impurities having a higher boiling point than aniline, to obtain, after phase separation, an organic phase and an aqueous phase, wherein alkali metal hydroxide is added to the aqueous phase and this aqueous phase is used as a constituent of the extractant used in step a); and
f.2) concentrating the organic phase from step e.2) by evaporation of aniline to obtain an aniline-containing distillate stream and a bottom stream containing organic impurities having a higher boiling point than aniline.

4. The process as claimed in claim 2, in which the extraction step is conducted in parallel with the acid treatment step.

5. The process as claimed in claim 1, in which step c):
comprises a distillation in a distillation column with side draw, wherein the stream of purified aniline is withdrawn as side stream, the gaseous stream containing organic impurities having a lower boiling point than aniline is withdrawn as top stream, and the liquid stream containing aniline and organic impurities having a higher boiling point than aniline is withdrawn as bottom stream from the distillation column with side draw;
or
comprises a distillation in two distillation columns connected in series, wherein in the first distillation column the gaseous stream containing organic impurities having a lower boiling point than aniline is withdrawn as top stream, wherein the bottom stream obtained in the first distillation column is conducted as feed into the second distillation column, wherein in the second distillation column the stream of purified aniline is obtained as top stream and the liquid stream containing aniline and organic impurities having a higher boiling point than aniline is obtained as bottom stream;
or
comprises a distillation in three distillation columns, wherein a top stream containing water and organic impurities is withdrawn from a first distillation column and is separated into an aqueous and an organic phase, the organic phase being conducted into a second distillation column from which the gaseous stream containing organic impurities having a lower boiling point than aniline is withdrawn as top stream, and wherein the bottom stream obtained in the first distillation column is conducted as feed into a third distillation column, wherein in the third distillation column the stream of purified aniline is obtained as top stream and the liquid stream containing aniline and organic impurities having a higher boiling point than aniline is obtained as bottom stream.

6. The process as claimed in claim 5, in which the gaseous stream containing organic impurities having a lower boiling point than aniline is partially liquefied, the liquid phase thus obtained, optionally after removal of any water present, is recycled into that distillation column from which the gaseous stream containing organic impurities having a lower boiling point than aniline was withdrawn as top stream, and wherein the gaseous phase remaining after the partial liquefaction is discharged from the distillation.

7. The process as claimed in claim 2, in which the gaseous phase remaining in step c) after the partial liquefaction is condensed, and the condensate obtained is used as an additional constituent of the organic stream to be used in step f.1).

8. The process as claimed in claim 2, further comprising, after the phase separation, incinerating the the organic phase obtained in step f.1).

9. The process as claimed in claim 2, in which the alkali metal salt different from an alkali metal hydroxide is an alkali metal chloride and the acid used in step e.1) is hydrochloric acid.

10. The process as claimed in claim 3, in which the alkali metal salt different from an alkali metal hydroxide is an alkali metal sulfate and the acid used in step d.2) is sulfuric acid.

11. The process as claimed in claim 1, in which the crude aniline fraction is obtained by hydrogenation of nitrobenzene in the presence of a catalyst and removal of water formed in the hydrogenation.

12. The process as claimed in claim 11, in which the hydrogenation of nitrobenzene is conducted in the gas phase and the gaseous reaction product obtained is condensed, wherein
(i) the condensation of the gaseous reaction product is conducted in two stages with successively falling temperature and only after the second condensation stage a phase separation for removing water formed in the hydrogenation takes place,
or
(ii) the condensation of the gaseous reaction product is conducted in one stage and following the condensation a phase separation for removing water formed in the hydrogenation takes place, and wherein the organic phase remaining after removal of the water is used as crude aniline stream in step a).

13. The process as claimed in claim 12, comprising case (i), in which
(i-1) the condensate obtained in the first condensation stage is combined with the organic phase remaining after the removal of water in the phase separation after the second condensation stage and is used as crude aniline stream in step a),
or
(i-2) the crude aniline stream to be provided in step a) is withdrawn either from the condensate obtained in the first condensation stage or from the organic phase remaining after the removal of water in the phase separation after the second condensation stage.

14. The process as claimed 1, in which the alkali metal of the alkali metal hydroxide and of the alkali metal salt different from an alkali metal hydroxide is in each case sodium or potassium.

15. The process as claimed in claim 14, in which
the alkali metal hydroxide used is sodium or potassium hydroxide in a concentration range from 0.009% by mass to 2.05% by mass, and
wherein the alkali metal salt used which is different from the alkali metal hydroxide is either
(1) sodium chloride or potassium chloride in a concentration range from 4.00% by mass to 25.0% by mass, or
(2) sodium sulfate or potassium sulfate in a concentration range from 2.40% by mass to 25.0% by mass.

16. The process as claimed in claim 3, in which the extraction step is conducted in parallel with the acid treatment step.

17. The process as claimed in claim 3, in which the gaseous phase remaining in step c) after the partial liquefaction is condensed, and the condensate obtained is used as an additional constituent of the organic stream to be used in step e.2).

18. The process as claimed in claim 3,further comprising, after the phase separation, incinerating the bottom stream obtained in step f.2).

19. The process as claimed in claim 2, in which the alkali metal salt different from an alkali metal hydroxide is an alkali metal sulfate and the acid used in step e.1) is sulfuric acid.

20. The process as claimed in claim 3, in which the alkali metal salt different from an alkali metal hydroxide is an alkali metal chloride and the acid used in step d.2) is sulfuric acid.
